(19) United States
(12) Patent Application Publication
(10) Pub. No.: US 2006/0217446 A1
(43) Pub. Date: Sep. 28, 2006

(54) Method for preventing and/or treating trichomonas vaginitis

(75) Inventors: Lei Huang, Duluth, GA (US); Shu-Ping Yang, Alpharetta, GA (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 11/091,206

(22) Filed: Mar. 28, 2005

(51) Int. Cl. A61K 31/045; C12Q 1/18

(52) U.S. Cl. 514/724; 435/32

(57) ABSTRACT

The invention provides a method for treating and/or preventing vaginal infections, such as trichomonas vaginitis, by administering a sugar alcohol-polyol to a subject in need of treatment. Particularly suitable sugar alcohol-polyols are pentitol and hexitol polyols, such as xylitol, arabitol/arabinitol, adonitol, ribitol, glycerol, dulcitol, inositol, mannitol and sorbitol. A composition containing a sugar alcohol-polyol for treating vaginal infections is also disclosed. It has been found that sugar alcohol-polyols are capable of selectively inhibiting the growth of Trichomonas vaginalis, a parasite that causes one of the most common vaginal infections, trichomonas vaginitis, while not inhibiting the growth of Lactobacillus acidophilus, the dominant bacteria in a healthy vaginal ecosystem. Sugar alcohol-polyols are safe and cost-effective, and can be used alone, with other polyols or incorporated into different vaginal health products to treat and/or prevent trichomonas vaginitis.

### BACKGROUND OF THE INVENTION

The vaginal ecosystem is a finely balanced environment maintained by a complex interaction among vaginal flora. A variety of bacteria, yeasts and other micro-organisms occur naturally in the vagina's environment. Lactobacillus acidophilus is the dominant bacteria in a healthy vaginal ecosystem, and it maintains an acidic environment of the vagina through the production of lactic acid. Lactic acid and hydrogen peroxide produced by Lactobacilli are toxic to anaerobic bacteria and other pathogenic bacteria in the vagina. The vaginal balance can be upset by external factors such as antibiotics, stress, illness and hormonal changes, and insults that decrease Lactobacilli result in an in overgrowth of pathogenic organisms in the vagina.

More than 75% of women will have at least one vaginal infection in their lives, and 50% of these women will have a recurrence of the infection (http://www.stopgettingsick.com/templates/news_template.cfm/1671). It has been reported that in the United States alone, about 13 million women experience vaginal infections each year.

Trichomonas vaginitis, also known as Trichomoniasis or trich, is one of the most common vaginal infections and this infection is considered to be a sexually transmitted disease. In the United States, it is estimated that more than 2 million women are infected each year.

Trichomonas vaginitis causes vulvar itching and an odorous vaginal discharge. It is caused by Trichomonas vaginalis, a single-celled protozoan parasite not normally found in the flora of the genitourinary tract. Trichomonas vaginalis is a flagellate protozoa that is pear-shaped and about the size of a white blood cell. These motile cells have four flagellae and a single nucleus.

This pathology is generally treated with an antibiotic such as metronidazole. This can be administered orally or vaginally. Metronidazole exhibits serious side effects, particularly on the blood and on the central nervous system, so much that in certain types of patients it has been necessary to discontinue the treatment, and authorities in the medical field have recommended that women who use metronidazole should not breast feed (Martindale, The Extra Pharmacopoeia, 29th Edition, 1989, page 667). Additionally, studies in rats and mice have provided some evidence that metronidazole may cause tumors in these species when administered orally for a long period at high doses. The relevance of these findings in humans is not known. It is therefore recommended that the use of metronidazole for the treatment of trichomoniasis should be confined to only those patients in whom significant Trichomonas vaginalis infection has been confirmed by appropriate diagnostic techniques.

There is therefore a need for a suitable compound or composition that can treat and/or prevent vaginal infections without killing the useful bacteria and without the side effects of known treatments.

### SUMMARY OF THE INVENTION

In response to the problems discussed above, it has been found that sugar alcohol-polyols are capable of selectively inhibiting and/or killing pathogens such as Trichomonas vaginalis without affecting Lactobacilli growth. These sugar alcohol-polyols are therefore suitable for use as active ingredients in a method of treating and/or preventing vaginal infections, and in particular, trichomonas vaginitis.

The polyols may be pentitols (5-carbon) or hexitols (6-carbon) compounds, such as (but not limited to) xylitol, arabitol/arabinitol, adonitol, ribitol, glycerol, dulcitol, inositol, mannitol and sorbitol.

According to a first aspect of the invention, a method of treating and/or preventing a vaginal infection using a sugar alcohol-polyol is described. The sugar alcohol-polyol may be a pentitol or hexinol, such as xylitol, arabitol/arabinitol, adonitol, ribitol, glycerol, dulcitol, inositol, mannitol and sorbitol. More particularly, the sugar alcohol-polyol may be a pentitol, such as xylitol, arabitol/arabinitol, adonitol and/or ribitol. Most particularly, the sugar alcohol-polyol is xylitol.

The sugar alcohol-polyol may be in the form of a solution, a powder and/or a crystalline structure. The sugar alcohol-polyol may be used alone, in combination with one or more other polyols or in a therapeutic amount in a composition, in the form of a foam, a cream, a gel, a jelly, a moisturizer, a spray, a suppository, a vaginal capsule, a vaginal tablet, a vaginal film, a vaginal sponge, a vaginal ovule or any other vaginal health product. The composition may also be applied to a vaginal insert, tampon, wipe or pad. The composition may further include a suitable diluent, excipient and/or auxiliary

In general, the sugar alcohol-polyol is present in the composition in an amount of from about 0.1 to about 20 percent (grams/100 milliliters (wt/vol)), more preferably in an amount of from about 1 to about 10 percent (wt/vol), even more preferably in an amount of from about 3 to about 7.5 percent (wt/vol), and even more preferably in an amount of about 5 percent (wt/vol).

The method comprises the step of administering the composition topically to a subject in need thereof, so as to inhibit the growth of Trichomonas vaginalis without inhibiting the growth of Lactobacillus acidophilus.

According to a second aspect of the invention, a composition for treating and/or preventing a vaginal infection is described. The composition comprises a therapeutically effective amount of at least one sugar alcohol-polyol and is substantially as described above.

According to a third aspect of the invention, the use of at least one sugar alcohol-polyol in a method of manufacturing a medicament for treating and/or preventing a vaginal infection is described.

### BRIEF DESCRIPTION OF THE DRAWINGS

FIG. 1 shows the effects of xylitol on Trichomonas vaginalis (OD) after 24 hours treatment at concentrations of 0.5 percent, 3.0 percent and 5.0 percent;

FIG. 2 shows the effects of xylitol on Trichomonas vaginalis (OD) after 48 hours treatment at concentrations of 0.5 percent, 3.0 percent and 5.0 percent; and

FIG. 3 the effects of xylitol on Lactobacillus acidophilus (OD) after 2, 4, 6, and 24 hours treatment (n=4).

### DETAILED DESCRIPTION OF THE INVENTION

The invention provides a method for treating a vaginal infection by topically administering a therapeutic amount of a sugar alcohol-polyol to a subject in need thereof. Sugar alcohol-polyols are polyols that are formed from, and can be converted to, sugars (i.e. aldoses and ketoses). These polyols, such as xylitol, can selectively inhibit and/or kill pathogens like Trichomonas vaginalis, without affecting Lactobacilli growth, the presence of the former being a cause of trichomonas vaginitis and the latter being a desirable presence in the ecosystem of the vagina.

Sugar alcohol-polyols display many common characteristics. For example, they are able to form complexes with calcium and certain other polyvalent cations; they facilitate the absorption of calcium through the gut wall; and they act as stabilizers of salivary calcium and phosphate ions. Apart from xylitol, other suitable sugar alcohol-polyols are the pentose-based and hexose-based polyols, such as (but not limited to) arabitol/arabinitol, adonitol, ribitol, glycerol, dulcitol, inositol, mannitol and sorbitol, and more particularly, arabitol/arabinitol, adonitol and ribitol. The invention will be described in further detail with reference to xylitol as an example of a sugar alcohol-polyol.

Xylitol is a five-carbon sugar polyol, small amounts of which occur naturally in plums, strawberries and raspberries. It has also been called “birch sugar”, as it can be produced from xylan derived from birch wood chips. It is equal in sweetness to sucrose, with 1 g yielding 4.06 kcal.

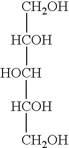

### Chemical Structure of Xylitol

Xylitol has been known since the late Ninteenth Century. German and French researchers were the first to produce xylitol about 100 years ago, when a syrup-like mixture was made. However, xylitol was not manufactured in a crystalline form until World War Two, and its status remained that of a research compound until it was used as an alternate sweetener during World War Two, due to war-associated sugar shortages.

The discovery of xylitol's insulin-independent properties resulted in it being introduced into diabetic diets, which was its primary use up until about 1975, when xylitol was first used as a sugar-free chewing gum. Since then, xylitol's other biological properties have been continually explored. There is increasing global awareness of xylitol's significant dental benefits (http://herkules.oulu.fi//isbn9514267796. Terhi Tapiainen (2002) Microbiological Effects And Clinical Use Of Xylitol In Preventing Acute Otitis Media), and the compound is widely used as a sweetener in sugar-free candy, gums and mints. Xylitol is believed to be a safe compound, and high levels of it are to be found in dental products such as toothpaste and chewing gums.

Xylitol is a normal intermediate of human metabolism, and several grams of it are produced daily by the liver (Terhi Tapiainen; Ylikahri, R. (1979) Metabolic and nutritional aspects of xylitol. Adv Food Res 25:159-80). Exogenous xylitol is metabolized to glucose and glucogen or pyruvate and lactate in the liver. Many bacteria are nevertheless unable to utilize xylitol as an energy source, and its presence is harmful to some bacteria despite the availability of an alternative energy source such as glucose (Assev, S., Vegarud, G., Rölla, G. (1980) Growth inhibition of Streptococcus mutans strain OMZ 176 by xylitol. Acta Path Microbiol Scand, 88:61-63; Knuuttila, M. L., Mäkinen, K. (1975) Effect of xylitol on the growth and metabolism of Streptococcus mutans. Caries Res, 9:177-89). More recently, xylitol has also been described for use in reducing ionic strength and activating endogenous antimicrobials to treat cystic fibrosis (U.S. Pat. No. 6,716,819 to Welsh and Zabner), mucosal yeast infections (U.S. Pat. No. 6,414,035 to Munita et al.) and respiratory infections (U.S. Pat. No. 6,066,677 to Uhari and Kontiokari).

It has been shown in several studies that xylitol significantly reduces the growth of Streptococcus mutans in the presence of glucose or sucrose (Assev, S. et al. (1980); Knuuttila, M. L. et al.; Edwardsson, S., Birkhed, D., Mejare, B. (1977) Acid production from lycasin, maltitol, sorbitol and xylitol by oral streptococci and lactobacilli. Acta Odontol Scand, 35:257-263; Assev, S., Waler, S. M., Rölla, G. (1983) Further studies on the growth inhibition of some oral bacteria by xylitol. Acta Path Immunol Scand, 91:261-265; Vadeboncoeur, C., Trahan, L., Mouton, C., Mayrand, D. (1983) Effect of xylitol on the growth and glycolysis of acidogenic oral bacteria. J. Dent Res, 62:882-884). Xylitol has also been shown to reduce the growth of Streptococcus salivarius, Streptococcus sanguis, Lactobacillus casei and some strains of Escherichia coli, Saccharomyces cerevisae and Salmonella typhii although its effect on these bacteria is modest in comparison to its effect on S. mutans (London, J., Hausman, S. (1982) Xylitol-mediated transient inhibition of ribitol utilization by Lactobacillus casei. J Bacteriol, 150:657-661; Reiner, A. M. (1977) Xylitol and D-arabitol toxicities due to derepressed fructose, galactitol, and sorbitol phosphotransferases of Escherichia coli. J Bacteriol; 132:166-173; Mäkinen, K., Söderling, E. (1981) Effect of xylitol on some food-spoilage micro-organisms. J Food Sci, 46:950-951; Macfadyen, L. P., Dorocicz, I. R., Reizer, J., Saier, M. H., Jr., Redfield, R. J. (1996) Regulation of competence development and sugar utilization in Haemophilus influenzae Rd by a phosphoenolpyruvate:fructose phosphotransferase system. Mol Microbiol, 21:941-952).

The microbiological mechanism of the action of xylitol has not yet been fully discovered. The most detailed study found in the literature relates only to S. mutans. This study showed that xylitol can be transported into S. mutans, where it is phosphorylated through a constitutive fructose phosphotransferase system. The phosphotransferase system in bacteria regulates many metabolic processes and the expression of various genes (Saier M H, Jr., Reizer J (1994) The bacterial phosphotransferase system: new frontiers 30 years later. Mol Microbiol 13:755-764). It is thus likely that xylitol can retard or inhibit bacteria growth through disturbing the metabolic processes in viable bacteria. It was also found that even very low concentrations of xylitol can damage the ultrastructure of viable S. mutans bacteria (Tuompo, H., Meurman, J. H., Lounatmaa, K., Linkola, J. (1983) Effect of xylitol and other carbon sources on the cell wall of Streptococcus mutans. Scand J Dent Res; 91:17-25); and their protein synthesis is also disturbed, which implies that xylitol acts as a strong metabolic inhibitor for this species. Xylitol also affects polysaccharide synthesis in S. mutans, resulting in decreased bacterial adherence (Söderling, E., Alaräisänen, L., Scheinin, A., Mäkinen, K. K. (1987) Effect of xylitol and sorbitol on polysaccharide production by and adhesive properties of Streptococcus mutans. Caries Res 21:109-116). Since bacteria adhere to host cells through carbohydrate-binding proteins (Ofek, I., Sharon, N. (1990) Adhesins as lectins: specificity and role in infection. Curr Top Microbiol Immunol, 151:91-11), extracellular xylitol may disturb the binding process by acting as a receptor analogue for the host cell, which could result in decreased adherence (Soderling et al. (1987); Kontiokari, T., Uhari, M., Koskela, M. (1998) Antiadhesive effects of xylitol on otopathogenic bacteria. J Antimicrob Chemother, 41 :563-565).

The applicant has now surprisingly found that xylitol has the effect of selectively inhibiting the parasite Trichomonas vaginalis while not inhibiting Lactobacillus acidophilus, and it is therefore a suitable compound for treating and/or preventing vaginal infections, and in particular, trichomonas vaginitis.

The sugar alcohol-polyol can be used in the form of a solution, powder and/or crystalline structure, either alone, in combination with another polyol or as part of a composition. It is typically administered topically as part of a composition which is in the form of a foam, cream, gel, jelly, moisturizer, spray, suppository, vaginal capsule, vaginal tablet, vaginal film, vaginal sponge, vaginal ovule or any other vaginal health product. The composition optionally also includes suitable diluents, excipients and/or auxiliaries, which are well known in the art.

The composition can be applied to a vaginal insert, tampon, wipe or pad or can be used on its own.

In general, the polyol is present in the composition in an amount of from about 0.1 to about 20 percent (grams/100 milliliters (wt/vol)), more preferably in an amount of from about 1 to about 10 percent (wt/vol), even more preferably in an amount of from about 3 to about 7.5 percent (wt/vol), and even more preferably in an amount of about 5 percent (wt/vol).

The present invention is further described by the following examples. Such examples, however, are not to be construed as limiting in any way either the spirit or scope of the invention.

### EXAMPLES

Microorganisms and Culture Media:

A sample of Trichomonas vaginalis, the parasite found in trichomonas vaginitis, was obtained from the American Type Culture Collection (ATCC), catalog number 30001. The culture medium was LYI-S-2 medium (ATCC medium 2154).

A sample of Lactobacillus acidophilus, a desirable bacterium in the vaginal ecosystem, was also obtained from the American Type Culture Collection (ATCC), catalog number 4354, and was cultured in ATCC medium 416.

### Example 1

### Effect of Xylitol on the Growth of Trichomonas vaginalis in Solution

A sterile LYI-S-2 medium was prepared according to the manufacturer's instructions, and the pH of this medium was adjusted to pH 6.0 using 1 N HCl. Xylitol was used as an example of a sugar alcohol-polyol. The xylitol, at concentrations of 0.5 percent, 3.0 percent and 5.0 percent, was dissolved into the LYI-S-2 medium and 0.9 milliliter of xylitol or culture medium only (as control) was added into different culture tubes.

0.1 milliliter of Trichomonas vaginalis culture suspension, at a concentration of 1×10⁶/milliliter, was added to each of the culture tubes, which were then incubated at 35 degrees Celsius on a 15 degree horizontal slant.

The viable Trichomonas vaginalis cells in each tube were counted under a microscope after 24 hours and 48 hours.

The above procedure was repeated four times for each concentration of xylitol and the control.

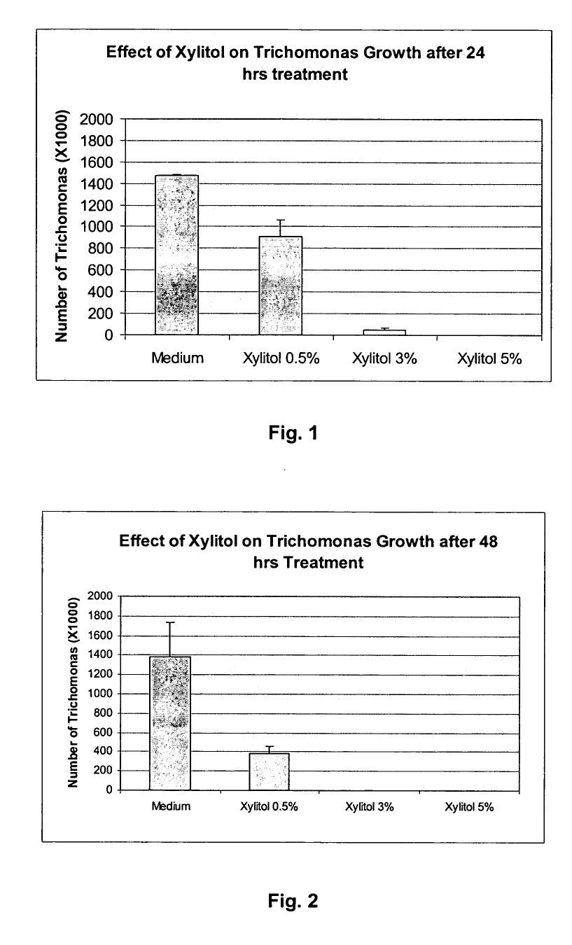

The results in FIG. 1 show that xylitol at concentrations of 0.5 percent, 3.0 percent and 5.0 percent significantly reduced the Trichomonas vaginalis cell count after 24 hours, when compared to the control group.

After 48 hours, xylitol was shown to have an even more significant inhibitory effect on the Trichomonas vaginalis cell count (FIG. 2). No live Trichomonas vaginalis cells were observed in the 3 percent and 5 percent xylitol treatment groups, while around 1.6 million Trichomonas vaginalis cells were counted in the control group.

### Example 2

### Effect of Xylitol on the Growth of Lactobacillus Acidophilus—Zone-of-Inhibition Test

A microorganism culture of 10⁵ cfu (colony forming units)/milliliter in a 1× phosphate buffered saline (PBS) solution (diluted from 10× PBS LIQUID CONCENTRATE from VWR Cat. No. EM-6507] was used. One milliliter of the solution was plated on proper agar plates, which were then incubated at 35 degrees Celsius for four hours. Three 4 millimeter diameter wells were punched into each agar plate. A test sample of 100 microliters of 5 percent xylitol in sterilized 2-N-morpholino ethane sulfonic (MES, pH=4.7) buffer (0.1 M 2-[morpholino]-ethanesulfonic acid, 0.9 percent NaCl, pH 4.7, prepared from BupH™ MES Buffer Saline Pack from Cat. No. 28390, Pierce Biotechnology, Inc., Rockford, Ill.) was added to one well of each plate. Into each of the other two wells were added MES buffer and 1 percent Benzyl Quats (diluted from BARDAC® 205M, from Lonza Inc., Fair Lawn, N.J.) as negative and positive controls, respectively. The plates were incubated overnight at 35 degrees Celsius. The presence of a zone of microorganism inhibition was measured the following day for Lactobacillus acidophilus activity.

As shown in Table 1, xylitol at a concentration of 5 percent did not affect the growth of Lactobacillus acidophilus. The positive control, 1 percent Benzyl Quats, inhibited the microorganism, while MES buffer itself had no effect on the microorganism.

TABLE 1
Effect of xylitol on Lactobacillus acidophilus
with zone inhibition test, n = 2.

| Tested Compounds/Polymers | Lactobacillus acidophilus |
|---|---|
| 5 percent xylitol | 0 mm |
| 1 percent Benzyl Quats | 15 mm |
| MES buffer | 0 mm |

### Example 3

### Effect of Xylitol on the Growth of Lactobacillus acidophilus in Solution Measured by Optical Density

Solutions having different concentrations of xylitol were prepared in culture media and sterilized by filtering. Control or xylitol solutions (0.9 milliliters) were added into culture tubes, and to this was added 0.1 milliliter of Lactobacillus acidophilus suspension at a concentration of around 10⁶ cfu/milliliter. The culture tubes were then incubated overnight at 37 degrees Celsius, whereafter the optical density was measured at 2, 4, 6 and 24 hours at 590 nanometers, by pippeting 100 microliters of the control or sample solutions into 96-well microplates, and then using a ThermoMax Microplate Reader from Molecular Devices of Sunnyvale, Calif., to obtain the optical density readings at 600 nm wavelengths.

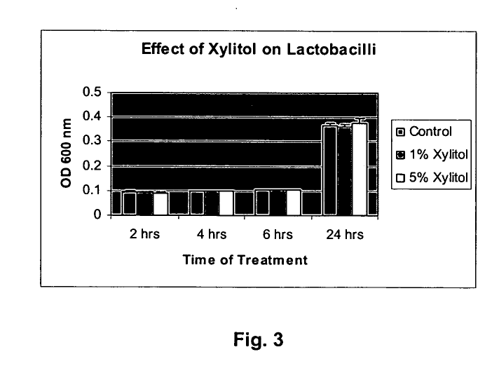

The results are depicted in FIG. 3 and clearly show that xylitol does not have an inhibitory effect on the growth of Lactobacillus acidophilus.

### Example 4

### Effect of Xylitol on the growth of Lactobacillus acidophilus in Solution Measured by Plate Counting

In this experiment, xylitol concentrations of 1 and 5 percent were tested on their effect on Lactobacillus acidophilus growth.

Xylitol solutions of 1 percent, 5 percent or control solutions (0.9 milliliters) were added into culture tubes, and to each tube was added a 0.1 milliliter Lactobacillus acidophilus suspension of around 10⁶ cfu/milliliter. The culture tubes were incubated at 37 degrees Celsius for 6 hours. The samples were then diluted at 1, 10 and 100 times and 100 microliters of each dilute sample was plated onto agar plates with WASP (Whitely Automatic Spiral Plate) spiral plating equipment, from Don Whitely Scientific Ltd, USA. The plates were incubated overnight at 35 degrees Celsius and the colonies were counted on each plate.

After 6 hours with either 1 percent xylitol or 5 percent xylitol, no significant inhibition on the growth of Lactobacillus acidophilus was observed (Table 2).

TABLE 2
Effect of xylitol on Lactobacillus acidophilus
after 6 hours treatment, n + 4

| Negative control | 1 percent xylitol | 5 percent xylitol |
|---|---|---|
| 3.85 ± 0.44E+05 | 3.77 ± 0.49E+05 | 3.97 ± 0.36E+05 |

The results of the above examples clearly demonstrate that different concentrations of a sugar alcohol-polyol are able to effectively inhibit Trichomonas vaginalis, without affecting the growth of Lactobacillus acidophilus. As sugar alcohol-polyols are naturally occurring, safe compounds which are also cost-effective, they are ideally suited to be formulated into vaginal health products, such as tampons, pads, wipes, vaginal moisturizers, sprays, gels and so forth for preventing and/or treating trichomonas vaginitis.

While the invention has been described in detail with respect to specific embodiments thereof, and in particular with respect to xylitol, it will be appreciated by those skilled in the art that various alterations, modifications and other changes may be made to the invention, such as the use of other sugar alcohol polyols, without departing from the spirit and scope of the present invention. It is therefore intended that the claims cover or encompass all such modifications, alterations and/or changes.

We claim:

1. A method of treatment of a trichomonas vaginitis infection which comprises administering topically to a subject in need of treatment a therapeutically effective amount of at least one sugar alcohol-polyol.

2. The method of claim 1, wherein the sugar alcohol-polyol is selected from the group consisting of pentitol and hexitol polyols.

3. The method of claim 1, wherein the sugar alcohol-polyol is selected from the group consisting of xylitol, arabitol, arabitinol, adonitol and ribitol.

4. The method of claim 1, wherein the sugar alcohol-polyol is part of a composition selected from the group consisting of a foam, a cream, a gel, a jelly, a moisturizer, a spray, a suppository, a vaginal capsule,.a vaginal tablet, a vaginal film, a vaginal sponge, a vaginal ovule, a vaginal insert, a tampon, a wipe and a pad.

5. The method of claim 1, wherein the sugar alcohol-polyol is in a form selected from the group consisting of a solution, a powder and a crystal structure.

6. The method of claim 1, wherein the treatment inhibits the growth of Trichomonas vaginalis and does not inhibit the growth of Lactobacillus acidophilus.

7. The method of claim 1, wherein the sugar alcohol-polyol is present in the composition in an amount of from about 0.1 to about 20 percent (wt/vol).

8. A method of preventing a trichomonas vaginitis infection which comprises administering topically to a subject in need of treatment a therapeutically effective amount of at least one sugar alcohol-polyol.

9. The method of claim 8, wherein the sugar alcohol-polyol is selected from the group consisting of pentitol and hexitol polyols.

10. The method of claim 8, wherein the sugar alcohol-polyol is selected from the group consisting of xylitol, arabitol, arabitinol, adonitol and ribitol.

11. The method of claim 8, wherein the sugar alcohol-polyol is part of a composition selected from the group consisting of a foam, a cream, a gel, a jelly, a moisturizer, a spray, a suppository, a vaginal capsule, a vaginal tablet, a vaginal film, a vaginal sponge, a vaginal ovule, a vaginal insert, a tampon, a wipe and a pad.

12. The method of claim 8, wherein the sugar alcohol-polyol is in a form selected from the group consisting of a solution, a powder and a crystal structure.

13. The method of claim 8, wherein the treatment inhibits the growth of Trichomonas vaginalis and does not inhibit the growth of Lactobacillus acidophilus.

14. The method of claim 8, wherein the sugar alcohol-polyol is present in the composition in an amount of from about 0.1 to about 20 percent (wt/vol).

15. The use of at least one sugar alcohol-polyol in a method of making a medicament for use in a method of preventing or treating a trichomonas vaginitis infection comprising administering topically a therapeutically effective amount of the at least one sugar alcohol-polyol to a patient in need thereof.

16. The use of a sugar alcohol-polyol according to claim 15, wherein the sugar alcohol-polyol is selected from the group consisting of pentitol and hexitol polyols.

17. The use of a sugar alcohol-polyol according to claim 15, wherein the sugar alcohol-polyol is selected from the group consisting of xylitol, arabitol, arabitinol, adonitol and ribitol.

18. The use of a sugar alcohol-polyol according to claim 15 wherein the sugar alcohol-polyol is in a form selected from the group consisting of a solution, a powder and a crystal structure.

19. The use of a according to claim 15, wherein the medicament is in the form of a sugar alcohol-polyol foam, a cream, a gel, a jelly, a moisturizer, a spray, a suppository, a vaginal capsule, a vaginal tablet, a vaginal film, a vaginal sponge, a vaginal ovule, a vaginal insert, a tampon, a wipe and a pad.

20. The use of a sugar alcohol-polyol according to claim 15, wherein the medicament is applied to a tampon, pad, wipe or vaginal insert.

21. The use of a sugar alcohol-polyol according to claim 15, wherein the sugar alcohol-polyol is present in the medicament in an amount of from about 0.1 to about 20 percent (wt/vol).

22. The use of a sugar alcohol-polyol according to claim 15, wherein the medicament inhibits the growth of Trichomonas vaginalis and does not inhibit the growth of Lactobacillus acidophilus.
